(19) United States
(12) Patent Application Publication
(10) Pub. No.: US 2005/0032116 A1
(43) Pub. Date: Feb. 10, 2005

(54) Analysis of insulin-like growth factors from biological fluids by the use of affinity-based mass spectrometric methods

(76) Inventors: Randall W. Nelson, Phoenix, AZ (US); Dobrin Nedelkov, Tempe, AZ (US); Urban A. Kiernan, Gilbert, AZ (US); Kemmons A. Tubbs, Mesa, AZ (US)

Correspondence Address: SNELL & WILMER;ONE ARIZONA CENTER, 400 EAST VAN BUREN, PHOENIX, AZ, 850040001, US

(21) Appl. No.: 10/756,100

(22) Filed: Jan. 12, 2004

Related U.S. Application Data
(60) Provisional application No. 60/439,110, filed on Jan. 10, 2003.

(51) Int. Cl. G01N 33/53; C12M 1/34

(52) U.S. Cl. 435/7.1; 435/287.2

(57) ABSTRACT

Presented herein are affinity-based mass spectrometric methods and assays for analysis of insulin like growth factors 1 and 2 (IGF-1 and IGF-2) present in complex biological mixtures and fluids. IGF-1 and IGF-2 were assayed from human plasma via BIA/MS, utilizing antibodies as ligands for affinity retrieval. Detection of both targeted and non-targeted IGFs in the mass spectra indicated possible protein complex retrieval by the individual antibodies. Plasma samples were investigated under variable denaturing conditions to confirm the detection of both free and bound IGFs. In a MSIA approach to IGF detection, pipettor tips containing porous solid supports covalently derivatized with anti-IGF antibodies were used to extract specific IGFs from plasma in preparation for mass spectrometry. Single or multiplex IGF-1 and IGF-2 assays were performed, resulting in detection of wild-type IGF-1 and 2, and a truncated IGF-2 variant, missing its N-terminal Alanine (also detected in the BIA/MS experiments). IGF-1 was quantified from several individuals via the use of internal reference standard species (rat IGF-1, doped into the samples prior to the MSIA analysis) and a working curve constructed from samples containing known concentrations of IGF-1.

### CROSS REFERENCE TO RELATED APPLICATION

This application claims the benefit of, and priority to, provisional application Ser. No. 60/439,110, filed Jan. 10, 2003, which application is hereby incorporated by reference in its entirety.

### FIELD OF THE INVENTION

The present invention is related to the field of proteomics and diagnostics and generally relates to the qualitative and quantitative characterization of insulin-like growth factors present in humans by use of mass spectrometry. More specifically, the present invention encompasses affinity capture methods and devices used for the selective retrieval of insulin like growth factor 1 (IGF-1) and insulin like growth factor 2 (IGF-2) from human biological fluids prior to mass spectrometric interrogation. The methods and devices can be used in stand-alone application with mass spectrometry, as in the case of Mass Spectrometric Immunoassay (MSIA), or can include methods of optical sensing during biomolecular recognition events, as in the case of Biomolecular Interaction Analysis Mass Spectrometry (BIA/MS). These methods and devices, which target IGF-1 or IGF-2 separately in individual assays, or both species in a simultaneous assay, find application in the clinical and diagnostic monitoring of the growth factors for the presence of interacting partners, qualitative changes brought on by genetic or posttranslational causes, or quantitave modulation due to disease or ailment.

### BACKGROUND OF THE INVENTION

With the recent first draft completion of the human genome, much attention is now shifting to the field of proteomics, where gene products (proteins), their variants, interacting partners and the dynamics of their regulation and processing are the emphasis of study. Such studies are essential in understanding, for example, the mechanisms behind genetic/environmentally induced disorders or the influences of drug mediated therapies, and are potentially becoming the underlying foundation for further clinical and diagnostic analyses. Critical to these studies is the ability to qualitatively determine specific variants of whole proteins (i.e., splice variants, point mutations and posttranslationally modified versions) and the ability to view their quantitative modulation.

Growth factors, in particular, are the subject of much study with regard to relating physiological changes (i.e., qualitative and quantitative modulation) to disease. Specifically, the insulin like growth factors 1 and 2 (IGF-1 and IGF-2), which are members of an important network of proteins that regulate metabolic, growth, and other cellular processes and activities, have been linked to abnormal growth, prostate cancer and breast cancer. Primarily synthesized in the liver, the IGFs circulate in serum in a form of protein complexes, bound to IGF-binding proteins (IGFBP). Less than 1% of the IGFs circulate in free, unassociated form. The binding to the IGFBPs increases the half-life of IGFs in blood, whereas the physiological role of the free IGF has not yet been determined. Structurally, IGF-1 and IGF-2 share 62% amino acid sequence homology, and there is 40% homology between the IGFs and proinsulin.

Immunoassays (ELISA, radio, or chemiluminescence) are generally used for assaying IGFs in plasma/serum. Because the concentration of free IGFs in serum samples can increase upon storage (due to proteases-induced release of the bound IGFs), determination of the total IGF is preferred in clinical research and practice. Acid ethanol extraction is commonly used to release the bound IGFs prior to assaying, although additional steps are often required to minimize the IGFBPs interference. IGFs measurements are routinely performed using commercially available immunoassays, and recently studies on large populations have yielded important correlations between increased IGF concentrations and the risk of cancer.

Although the conventional immunoassay approaches have found considerable use in the quantitative monitoring of the growth factors, they suffer from a common fault of all immunological assays that rely on the indirect detection of the species under investigation; that being the inability to readily differentiate between variants of the same protein. With regard to human beings, there are several possible causes for the presence of multiple and variable species of the same protein in individuals. These causes include, genetic heterozygosity, translational splice variation and/or variable posttranslational modifications. The two former causes require that any quantitative assay be accompanied by a second assay that is able to qualify (i.e., either confirm the wild-type or determine a mutant) gene sequence. Likewise, the latter cause requires an additional qualitative assay to confirm that the protein under investigation is in fact the “correct” form, i.e., of wild-type posttranslational modification. Thus, for absolute certainty, any immunological assay that utilizes indirect means of detection (e.g., secondary antibody conjugated to a fluorescent or radioactive reporter) must be accompanied by a second qualitative analysis able to unambiguously confirm or identify the exact (not presumed) protein species under investigation. Because of strict biological function—structure relationships, quantitative assays not accompanied by corresponding rigorous qualitative assay can in the least be erroneous, and, at worst, meaningless.

Moreover, there are several real-life challenges inherent to the analysis of the IGFs, and of all proteins in general. Foremost is the fact that any protein considered relevant enough to be analyzed resides in vivo in a complex biological environment or media. The complexity of these biological media present a challenge in that, oftentimes, a protein of interest is present in the media at relatively low levels and is essentially masked from analysis by a large abundance of other biomolecules, e.g., proteins, nucleic acids, carbohydrates, lipids and the like. In other instances, proteins are complexed tightly with other biomolecules that might interfere with their analysis. In order to analyze proteins of interest from- and in- their native environment, assays capable of assessing proteins present in a variety of biological fluids, both qualitatively and quantitatively, are needed. These assays must: 1) Be able to selectively retrieve and concentrate specific proteins/biomarkers from biological fluid for subsequent high-performance analyses, 2) Be able to quantify targeted proteins, 3) Be able to recognize variants of targeted proteins (e.g., splice variants, point mutations and posttranslational modifications) and to elucidate their nature, and 4) Be capable of analyzing for, and identifying, ligands interacting with targeted proteins.

Thus, there is a pressing need for new and improved technologies able to characterize insulin-like growth factors, both qualitatively and quantitatively, in a single assay. Likewise, there exists a pressing need for techniques that are able to readily study both unbound (i.e., free in solution) and bound (i.e., complexed with IGFBP and other proteins) insulin-like growth factors and associated components for use in the study of the biophysical properties of wild-type and variant forms of the growth factors. Two protein mass spectrometry techniques, matrix-assisted laser desorption/ionization time-of-flight mass spectrometry (MALDI-TOF MS) and electrospray ionization mass spectrometry, offer the particular advantages of detecting multiple proteins in the same analysis and being able to differentiate between mass-shifted variant forms of the same protein. Mass resolution of related protein species also allows mass-shifted variants of a target protein to be intentionally incorporated into the analysis for use as internal reference standards for quantitative analysis. In this manner, affinity capture assays can be designed where a single pan-antibody is used to retrieve all protein variants (and in vivo assembled complexes) from a biological fluid, upon which each variant or component is detected during mass spectrometry at a unique and characteristic molecular mass. Two recently developed technologies that use this affinity retrieval procedure in a combination with mass spectrometry for detection and characterization of proteins from complex biological fluids are Mass Spectrometric Immunoassay (MSIA) (Nelson, R. W., Krone, J. R., Bieber, A. L. and Williams, P. (1995) Anal. Chem. 67, 1153-1158; Niederkofler, E. E., Tubbs, K. A., Gruber, K., Nedelkov, D., Kiernan, U. A., Williams, P. and Nelson, R. W. (2001) Anal. Chem. 73, 3294-3299; Kiernan, U., Tubbs, K., Nedelkov, D., Niederkofler, E. and Nelson, R. (2002) Biochem. Biophys. Res. Commun. 297, 401; Tubbs, K. A., Nedelkov, D. and Nelson, R. W. (2001) Anal. Biochem. 289, 26-35)—an assay that is used for the unambiguous detection and rigorous quantification of polypeptides/proteins retrieved from complex biological systems, and Biomolecular Interaction Analysis Mass Spectrometry (BIA/MS) (Krone, J. R., Nelson, R. W., Dogruel, D., Williams, P. and Granzow, R. (1997) Anal. Biochem. 244, 124-32; Nelson, R. W., Krone, J. R. and Jansson, O. (1997) Anal. Chem. 69, 4363-8; Nelson, R. W., Nedelkov, D. and Tubbs, K. A. (2000) Anal. Chem. 72, 404A-411A; Nedelkov, D. and Nelson, R. W. (2000) J. Mol. Recogn. 13, 140-145; Nedelkov, D. and Nelson, R. W. (2001) Am. J. Kidney Dis. 38, 481-7)—which combines Surface Plasmon Resonance (SPR) quantification with mass spectrometry.

For the foregoing reasons, there is a need for MSIA and BIA/MS devices, kits, methods and protocols for the rapid and efficient qualitative and quantitative characterization of insulin-like growths factors, their phenotypic variants and their in vivo binding components.

Although the present invention has been described in some detail by way of illustration and example for purposes of clarity and understanding, it will be apparent that certain changes and modifications may be practiced within the scope of the invention.

### SUMMARY OF THE INVENTION

It is an object of the present invention to devise MSIA and BIA/MS methods that prepare IGF 1 and 2, in micro-sample form, directly from biological fluid to be used in detecting and quantifying the growth factors present in human plasma and serum.

It is another object of the present invention to construct devices, in the form of pipettor tips containing porous solid supports that are constructed, covalently derivatized with affinity ligand (termed MSIA-Tips), that extract IGF-1 and IGF-2, and their variants, from various biological fluids by repeatedly flowing the fluids through the MSIA-Tips.

It is yet another objective of the present invention to incorporate internal references species (IRS)—mass-shifted variants of the insulin-like growth factors—into analytical samples for co-extraction with the IGFs (in order to normalize sample extractions and data acquisition) for quantification of the growth factors.

It is still a further objective of the present invention to use either MSIA or BIA/MS in the protein phenotyping of individuals by detecting and identifying point mutations or posttranslational variants of the IGFs.

Yet another objective of the present invention is the development of multi-analyte assays capable of simultaneously characterizing both IGF-1 and IGF-2 in a single analysis.

It is still another objective of the present invention to use BIA/MS for both the optical and mass spectrometric characterization of insulin-like growth factors in either their native, in vivo environment or in denaturing conditions

A further object of the present invention enables useful product kits for the characterization of insulin-like growth factors directly from biological fluids for linkage and correlation to disease.

The present invention includes the ability to selectively retrieve and concentrate insulin-like growth factors from biological fluid for subsequent high-performance analyses (e.g. MALDI-TOF MS), the ability to identify targeted biomolecules, the ability to quantify targeted biomolecules, the ability to recognize variants of targeted biomolecules (e.g., splice variants, point mutations and posttranslational modifications) and to elucidate their nature, and the capability to analyze for, and identify, ligands interacting with targeted biomolecules. The invention itself, both as to its structure and its operation together with the additional objects and advantages thereof will best be understood from the following description of the preferred embodiment of the present invention when read in conjunction with the accompanying drawings. The preferred embodiment of the invention is described bellow in the Drawings and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

### BRIEF DESCRIPTION OF THE DRAWINGS

FIG. 1 is an illustration of the BIA/MS procedure.

FIG. 2 shows SPR sensorgrams resulting from the immobilization of anti-IGF-1 and anti-IGF-2 antibodies onto FC 1 and FC2 of the biosensor chip, respectively.

FIG. 3 shows SPR sensorgrams obtained by flowing human plasma (50-fold dilution) over FC1 and FC2 under non-denaturing conditions.

FIG. 4 shows MALDI-TOF MS spectra taken directly from FC1 and FC2 after exposure to 50-fold diluted human plasma under non-denaturing conditions.

FIG. 5 shows MALDI-TOF MS spectra taken directly from anti-IGF-1 and anti-IGF-2 derivatized flow cells after exposure to 10-fold diluted human plasma under non-denaturing conditions.

FIG. 6 shows comparative SPR sensorgrams resulting from flowing human plasma (10-fold dilution) over anti-IGF-1 and anti-IGF-2 derivatized flow cells in a) non-denaturing conditions, b) mildly denaturing conditions, and, c) strongly denaturing conditions.

FIG. 7 shows MALDI-TOF MS spectra taken directly from the anti-IGF-1 and anti-IGF-2 derivatized flow cells after exposure to the 10-fold diluted plasma under strongly denaturing conditions.

FIG. 8 is a schematic illustration of the MSIA procedure.

FIG. 9 is a comparison showing MALDI-TOF mass spectra of plasma, and IGF-1 and IGF-2 MSIA of the same plasma (mass range 4-80 kDa).

FIG. 10 is a comparison showing MALDI-TOF mass spectra of plasma, and IGF 1 and IGF-2 MSIA of the same plasma (mass range 6-9 kDa).

FIG. 11. MSIA spectrum of IGF-1 and IGF-2 (and a truncated variant) obtained from human plasma (40 μL) using a two-antibody MSIA-Tip (anti-IGF-1 and anti-IGF-2).

FIG. 12 Anti-IGF-1 MSIA applied to rat (rIGF-1) plasma, human (hIGF-1) plasma, and a human/rat plasma mixture. The rat IGF-1 is detected at m/z=7,686.88 Da, sufficiently resolved from the human IGF-1(m/z=7,649.7 Da) for use as an internal reference standard.

FIG. 13 Mass spectra obtained from eight standard sample, containing human IGF-1 (hIGF-1) in a concentration ranging from 0.008 to 1 μg/mL, and a constant amount of rat plasma, used in generating a standard working curve. All spectra are normalized (y-axis) to the rIGF-1 signal.

FIG. 14 Working curve relating the hIGF-1 concentration to normalized signal intensity. A linear relationship is observed for the concentration range from 0.008 to 1 μg/mL.

FIG. 15 Quantitative IGF-1 MSIA applied to eight individuals.

### DETAILED DESCRIPTION OF THE PREFFERED EMBODIMENTS

The present invention provides for methods, devices and kits for the BIA/MS analysis of insulin-like growth factors, their variants and binding partners present in various biological fluids.

Another embodiment of the present invention provides for methods used in the comparative and rigorous SPR quantification of IGFs and their variants present in various biological fluids.

Still another embodiment of the present invention provides for methods, devices and kits to be used in the MSIA analysis of insulin-like growth factors and their variants present in various biological fluids.

Yet another embodiment of the present invention provides for methods used in the MSIA quantification of IGFs and their variants present in various biological fluids.

Still yet another embodiment of the present invention enables the simultaneous detection and characterization of IGF-1 and IGF-2 in a single MSIA or BIA/MS assay.

Yet another embodiment of the present invention provides for the use of MSIA or BIA/MS in screening of individuals or large populations for IGFs and variants present in various biological fluids.

Specific embodiments in accordance with the present invention will now be described in detail using the following lexicon. These examples are intended to be illustrative, and the invention is not limited to the materials, methods or apparatus set forth in these embodiments.

As used herein, “MSIA-Tips” refers to a pipettor tip containing an affinity reagent.

As used herein, “affinity reagent” refers to a contiguous (formed/molded), porous, high surface area base support containing a low dead-volume (e.g. <2 μL of unused volume) to which affinity ligands are immobilized. The composition of the base support may be, but is not limited to, glasses, silica glasses, silica, silicon, plastics, polymers, metals, or any combination of these materials and the like. Affinity ligands are immobilized to the base support through the process of chemical activation.

As used herein “chemically activate” refers to the process of exposing the affinity reagent to chemicals in order to subsequently attach tethering linkers and/or affinity ligands. Compounds able to activate affinity reagents may be, but are not limited to organic or inorganic reagents. Often, it is advantageous to activate the affinity reagent base support using multiple steps including the use of a tethering linker. As used herein, “tethering linker” refers to compounds intermediate to the base support and the affinity ligand that exhibit the desirable characteristics of being able to be derivatized with high densities of affinity ligand and showing low binding of non-specified compounds. The tethering linker may be intrinsically active or require activation for attachment. Suitable tethering compounds include, but are not limited to, homo/hetero functional organics, natural and synthetic polymers, and biopolymers.

As used herein, “affinity ligand” refers to atomic or molecular species having an affinity towards analytes present in biological mixtures. Affinity ligands may be organic, inorganic or biological by nature, and can exhibit broad (targeting numerous analytes) to narrow (target a single analyte) specificity. Examples of affinity ligands include, but are not limited to, receptors, antibodies, antibody fragments, synthetic paratopes, enzymes, proteins, multi-subunit protein receptors, mimics, chelators, nucleic acids, and aptamers.

As used herein, “analyte” refers to molecules of interest present in a biological sample. Analytes may be, but are not limited to, nucleic acids, DNA, RNA, peptides, polypeptides, proteins, antibodies, protein complexes, carbohydrates or small inorganic or organic molecules having biological function. Analytes may naturally contain sequences, motifs or groups recognized by the affinity ligand or may have these recognition moieties introduced into them via chemical or enzymatic processes.

As used herein, “biological fluid” refers to a fluid or extract having a biological origin. Biological fluid may be, but are not limited to, cell extracts, nuclear extracts, cell lysates or biological products used to induce immunity or substances of biological origin such as excretions, blood, sera, plasma, urine, sputum, tears, feces, saliva, membrane extracts, and the like.

As used herein, “internal reference standard” (IRS) refers to analyte species that are modified (either naturally or intentionally) to result in a molecular weight shift from targeted analytes and their variants. The IRS can be endogenous in the biological fluid or introduced intentionally. The purpose of the IRS is that of normalizing all extraction, rinsing, elution and mass spectrometric steps for the purpose of quantifying targeted analytes and/or variants.

As used herein, “posttranslational modification” refers to any polypeptide alteration that occurs after synthesis of the chain. Posttranslational modifications may be, but are not limited to, glycosylations, phosphorylations, and the like.

As used herein, “mass spectrometer” refers to a device able to volatilize/ionize analytes to form vapor-phase ions and determine their absolute or relative molecular masses. Suitable forms of volatilization/ionization are laser/light, thermal, electrical, atomized/sprayed and the like or combinations thereof. Suitable forms of mass spectrometry include, but are not limited to, Matrix Assisted Laser Desorption/Time of Flight Mass Spectrometry (MALDI-TOF MS), electrospray (or nanospray) ionization (ESI) mass spectrometry, or the like or combinations thereof.

The following examples illustrate the analysis of IGF-1 and IGF-2 via BIA/MS and MSIA.

### EXAMPLE 1

### General BIA/MS

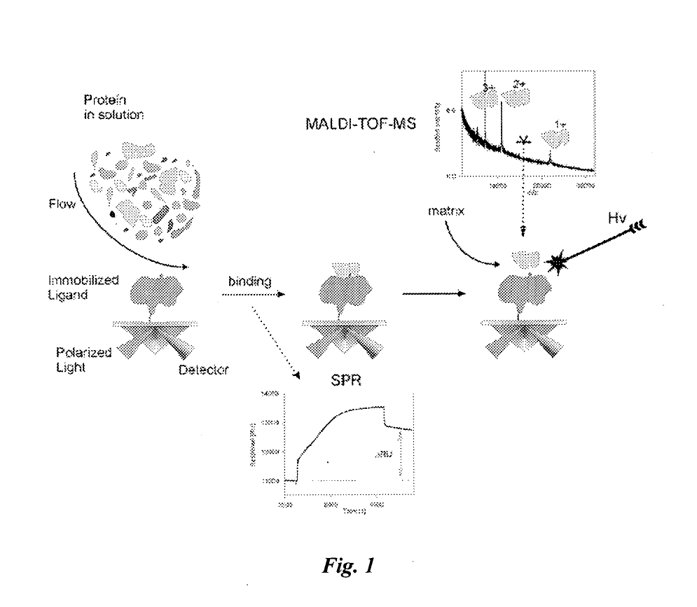

In its core, BIA/MS is a synergy of two individual technologies: surface plasmon resonance (SPR) sensing and matrix-assisted laser desorption/ionization time-of-flight. (MALDI-TOF) mass spectrometry (FIG. 1). Each technology brings a unique dimension to the BIA/MS analysis: SPR is employed for protein quantification, whereas MALDI-TOF MS is utilized to delineate structural features of the analyzed biomolecules. In the center of the BIA/MS analysis is a small chip compatible with and functional during SPR and MALDI-TOF MS. In the present BIA/MS configuration, the chip (a gold-coated glass slide) comes in liquid contact via microfluidics delivery system that forms highly defined sites on the chip surface. These sites are derivatized with a number of functional groups susceptible to covalent ligand attachment. Immobilization of ligand molecules to the sites is performed on-line, with SPR monitoring and facilitating the immobilization process. Analyte-containing samples are then delivered individually over the ligand-activated surfaces via the microfluidics delivery system, and the binding of the analyte to the immobilized ligand is quantified via SPR. The end result of the SPR analysis is a quantified amount of concentrated analyte(s) localized on precise locations on the chip surface. Because the SPR detection is non-destructive, the analytes (i.e., proteins) retrieved on the sensor chip during SPR can be MALDI-TOF MS analyzed from the very same surface where the interactions occurred. In such, the sensor surfaces used in the SPR experiments can be converted into amenable MALDI targets via minimal physical modifications and thorough application of a MALDI matrix. The chip is then subjected to MALDI TOF mass spectrometry, which yields the masses of the affinity-retained analytes and of other specifically or non-specifically bound biomolecules.

### EXAMPLE 2

### BIA/MS Chip Preparation for IGFs

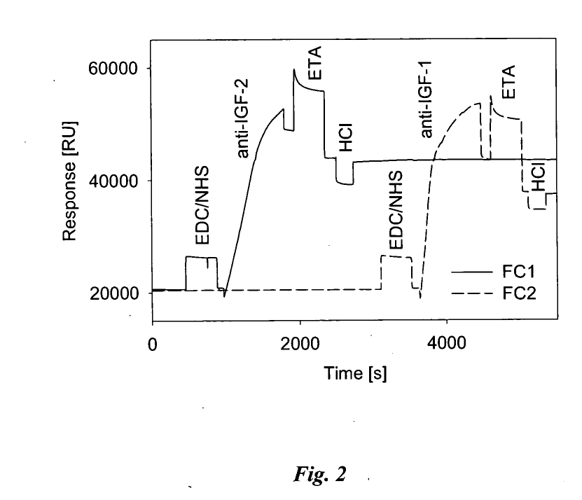

FIG. 2 shows the immobilization of anti-IGF-1 and anti-IGF-2 on the surface of flow cell 2 (FC2) and flow cell 1 (FC1) in the Biacore Biosensor, respectively. To start with, the carboxyl groups of the carboxymethyldextran matrix in the first flow cell were activated (converted to active esters) by a 35-μL injection of EDC/NHS. Next, a 70-μl aliquot of a solution of anti-IGF-2 (0.05 mg/mL, in 10 mM pH 5.0 acetate buffer) was injected. Following the coupling reaction, blocking of the free (unreacted) esters was achieved with a 35-μL injection of ethanolamine (ETA), followed by a 20-μL injection of 0.06 M HCl to release the non-covalently attached antibody. The SPR response (in resonance units, RU) measured at the end of the EDC/NHS injection was subtracted from the final SPR response measured after the HCl injection to yield an accurate estimate on the total amount of antibody immobilized on the surface of the flow cell. Because 1 RU equates to 1 picogram of proteinacious material per 1 mm² of the flow cell surface (the FC dimensions are 0.5×2 mm), the observed response of 22,350 RU indicates immobilization of 22.35 ng material on the surface of FC1, which corresponds to ˜149 fmole antibody (MW_{IgG}˜150,000). In similar manner, anti-IGF-1 was immobilized in the second (FC2) flow cell. A 70 μL of an anti-IGF-1 solution (0.025 mg/mL in 10 mM pH 5.0 acetate buffer) was injected over the EDC/NHS-activated flow cell surface. Following the ethanolamine and HCl injections, the SPR response of ΔRU=16,750 indicated ˜111 fmole anti-IGF-1 immobilized on the FC2 surface.

### EXAMPLE 3

### BIA/MS of IGFs from 50-Fold Diluted Plasma (Non-Denaturing Conditions)

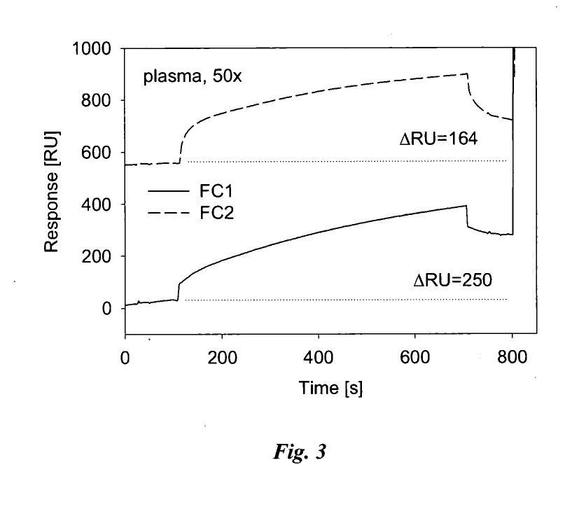

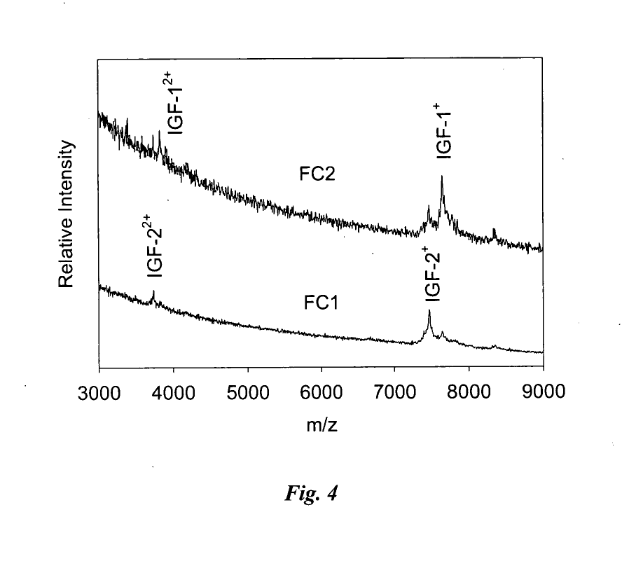

Following antibody immobilization as described in EXAMPLE 2, a 50 μL aliquot of fresh, 50-fold diluted human plasma was injected over the antibody-derivatized FC1 and FC2 surfaces (FIG. 3). At the time of chip undocking from the biosensor, responses of 250 and 164 RU were observed in FC1 and FC2, indicating binding of 250 and 164 pg of proteinaceous material, respectively. The mass spectra taken from the surfaces of the two flow cells after the plasma injection are shown in FIG. 4. Noticeable are signals (singly and doubly charged ions) coming from the targeted proteins: IGF-1 signals (MW_{IGF-1}=7648.7) dominate the spectrum obtained from the surface of FC2 (the anti-IGF-1 derivatized flow cell), whereas signals from IGF-2 (MW_{IGF-2}=7469.4) are observed in the mass spectrum taken from the FC1 surface (the anti-IGF-2 derivatized flow cell). Interestingly, smaller intensity signals from IGF-2 in FC2, and IGF-1 in FC1, are also present, even though they were not targeted by the corresponding antibodies in these flow cells. There are three possible explanation for the observance of these signals: 1) an analyte cross-walk occurred between the two flow-cells in the post-biosensor manipulation (most notably, the application of the MALDI matrix); 2) the immobilized antibodies exhibit cross reactivity toward the non-targeted protein (as already stated, the IGFs share 62% sequence homology; and 3) a protein complex containing both IGF-1 and IGF-2 was retrieved during the SPR analysis.

### EXAMPLE 4

### BIA/MS of IGFs from 10-Fold Diluted Plasma (Non-Denaturing Conditions): Investigation of Protein Complex Binding

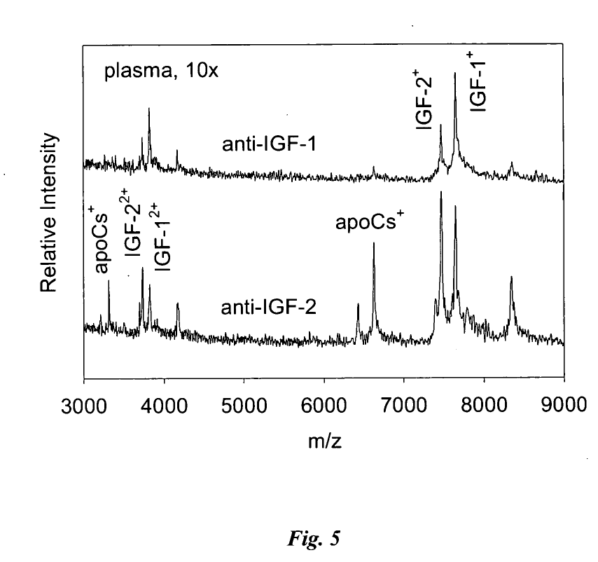

In order to eliminate the possibility of cross-walking between the adjacent flow cells, two additional CM5 chips was utilized: a single flow cell on the first chip was derivatized with anti-IGF-1, and one flow cell on the second chip was derivatized with anti-IGF-2. A 50 μL aliquot of fresh human plasma, diluted 10-fold, was injected over both chips in two separate experiments (sensorgrams not shown), and the chips were undocked and analyzed using MALDI-TOF MS. The resulting mass spectra are shown in FIG. 5. The presence the two IGFs in both mass spectra is clearly indicated by their corresponding signals, discounting the possibility of flow cell-cross-walking in the previous experiment. Moreover, due to the better resolution of the spectra, the signal at lower m/z from the main IGF-2 peak in the mass spectrum obtained from the anti-IGF-2 derivatized flow cell was identified as a truncated form of IGF-2 missing its N-temminal Alanine (MW=7,398.3). The spectra also contain several other signals, two of which can be attributed to apolipoprotein C-I (ApoC-I, MW=6,630.6) and its truncated isoform missing the N-terminal Thr-Pro residues (ApoC-I′, MW=6,432.4). Apo C-I and Apo C-I′ are abundant plasma proteins that bind non-specifically to the chip surface. This higher level of non-specific binding was somewhat expected due to the high concentration of plasma (10-fold) utilized in this example.

### EXAMPLE 5

### BIA/MS of IGFs from 10-Fold Diluted Plasma (Variable Denaturing Conditions): Investigation of Protein Complex Binding

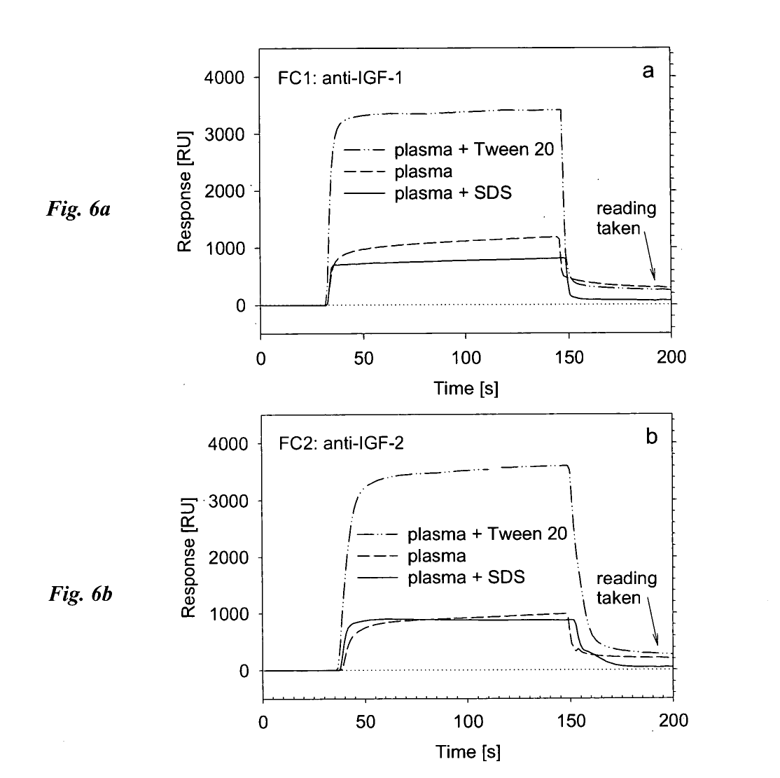

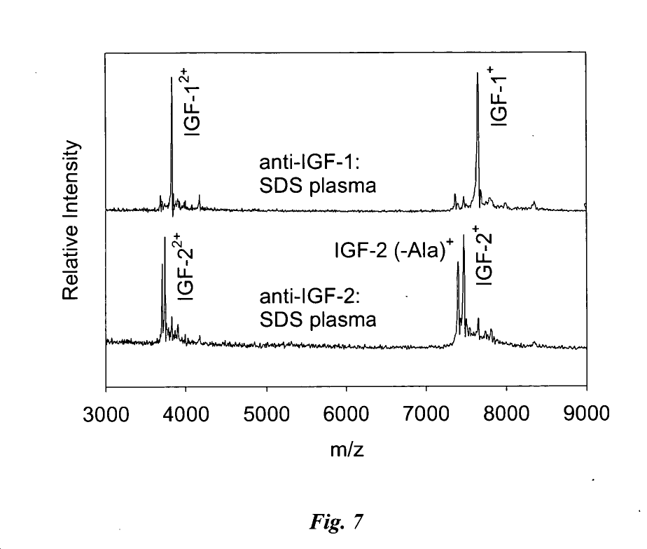

In order to more substantially demonstrate the retrieval of the protein complex, fresh human plasma was treated with several detergents to possibly disrupt the protein complex and release its constituent proteins. For the first sample, 20 μL of pure plasma (undiluted) was mixed with 20 μL of 0.5% SDS solution, incubated 30 min at room temperature, and further diluted with 160 μL of HBS-EP buffer to yield a plasma sample diluted 10-fold in buffer and 0.05% SDS. Another sample of plasma (10-fold diluted) was prepared in HBS-EP buffer containing 0.1% Tween 20. These two samples, along with a non-treated plasma control sample (10-fold diluted in HBS-EP) were injected in 10 μL aliquots over anti-IGF-1 and IGF-2 derivatized surfaces on a new CM5 sensor chip. The resulting sensorgrams are shown in FIG. 6. The injection of the SDS-treated plasma sample resulted in SPR responses of 80 and 51 RU in FC1 and FC2, respectively (the readings were taken 85 s after the end of the injections). These responses are significantly lower than the responses observed from the untreated sample injection (288 RU in FC1 and 197 RU in FC2), and the SPR responses observed after the injection of the Tween-treated plasma sample (239 RU in FC1 and 246 in FC2). The lower responses observed for the SDS-plasma sample could indicate the possible disruption of the protein complex and retrieval of only IGF-1 and IGF-2 by the immobilized antibodies, which would in turn yield lower SPR responses due to the lesser amount of total protein amount captured on the surface. In preparation for MALDI-TOF MS analysis, another aliquot of the SDS-treated plasma sample (50 μL) was injected over the regenerated surface of the same sensor chip, yielding SPR responses of 287 and 96 RU in FC1 and FC2, respectively (sensorgram not shown). The mass spectra taken from the surface of this sensor chip are shown in FIG. 7. The signals from the targeted proteins (IGF-1 in the anti-IGF-1 derivatized flow cell, and IGF-2 and its truncated isoform in the anti-IGF-2 FC) dominate the spectra (when compared with the results of the non-denaturing conditions approach; see FIG. 5), with very little presence of the other non-targeted IGF. The experimental data shown in EXAMPLES 2-5 strongly suggest that both bound and free IGF-1 and IGF-2 from human plasma were detected by using single antibodies, but with different sample preparation. Ligands with affinities toward a protein that is part of in-vivo assembled complexes can be used as “hooks” to retrieve the entire protein complex from a biological sample prepared under native (non-denaturing) conditions. In BIA/MS, the SPR sensing offers a unique opportunity to monitor the state of these protein complexes as a function of solvent variations, whereas the subsequent MALDI-TOF MS analysis of the retained components yields signals that reveal the masses of the constituent proteins, along with any structural modifications present. Given the dual aspect of the analysis (quantitative and qualitative), BIA/MS holds great promise in investigating protein complexes and the mechanisms behind their assembly.

### EXAMPLE 6

### General MSIA

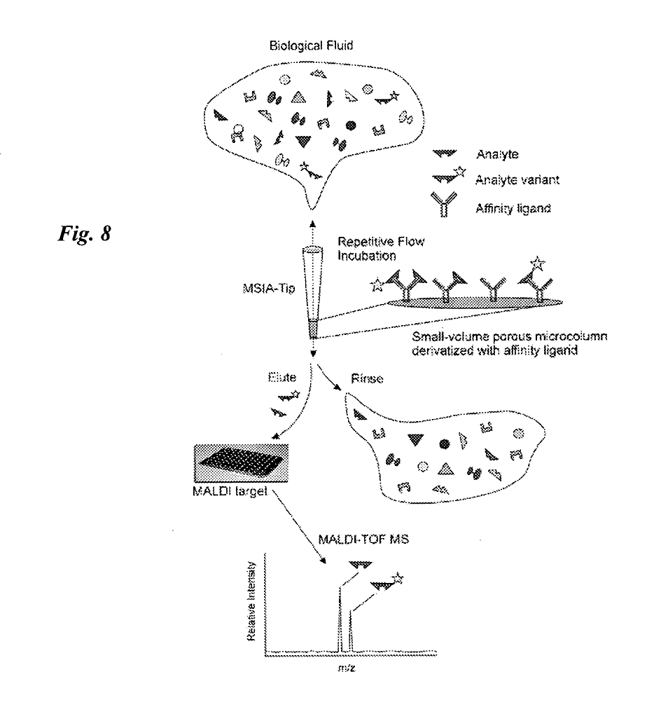

The general MSIA approach is shown graphically in FIG. 8. MSIA-Tips, containing porous solid supports covalently derivatized with affinity ligands are used to extract the specific analytes and their variants from biological samples by repetitively flowing the samples through the MSIA-Tips. Once washed of the non-specifically bound compounds, the retained analytes are eluted onto a mass spectrometer target using a MALDI matrix. MALDI-TOF MS then follows, with analytes detected at precise m/z values. The analyses are qualitative by nature but can be made quantitative by incorporating mass-shifted variants of the analyte into the procedure for use as internal standards.

### EXAMPLE 7

### Preparation of IGF MSIA-Tips

MSIA-Tips targeting IGF 1 and IGF 2 were prepared by covalently linking anti-IGF (1 or 2) antibodies onto frits contained within pipettor tip barrels. The frits were produced in bulk by loading soda lime glass beads into stainless steel annealing molds and baked to form a solid, yet porous frit. The frits were then removed and acid conditioned prior to a 12-hour treatment with 10% aminopropyl triethoxysilane. The amine-functionalized frits were then equilibrated in a phosphate buffer, after which it was replaced with a mixture of 15-kDa molecular mass carboxymethyl dextran (CMD), and N, N′-carbonyl diimidazole (CDI) to produce frits with surfaces covered with carboxyl groups. The carboxyl groups were activated, prior to antibody coupling, by vigorously rinsing away any free CMD with phosphate buffer and activating the carboxyl surface with an additional volume of CDI. The activated frits were loaded into wide-bore P-200 pipette tips and the tips were subsequently attached to a 96-format robotic pipetting workstation. In-robotic antibody coupling was performed by first flowing 100 μL of anti-IGF (1 or 2) antibody solution (0.1 mg/mL in 10 mM sodium acetate, pH 4.8) through the frits for approximately 40 minutes (by aspirating and dispensing 50 μL volumes). The remaining active sites of the frit were blocked with ethanolamine (1M, pH 8.5) and the tips were equilibrated in HBS buffer prior to their use. This process yielded affinity tips targeting the IGFs, which were found to be stable and active for a period of at least one month following antibody coupling (by storing at 4° C. in saline buffer).

### EXAMPLE 8

### Qualitative Analysis of IGF 1 and IGF 2 using MSIA (Individual Assays)

Individual samples for MSIA were prepared by mixing 40-μL aliquots of whole plasma with 60 μL of HEPES buffered saline solution (HBS) and 60 μL of a 0.05% SDS (w/v). The mixture was given adequate time (˜15 minutes) to disrupt all in vivo bound IGFs from their protein complexes, whereupon an additional 840 μL of HBS buffer was added to the solution. IGF-1 or IGF-2 was selectively extracted from the diluted, SDS-treated plasma by repeatedly aspirating and then expelling (˜50 times) 200 μL aliquots of solution through MSIA-Tips, derivatized with either anti-IGF-1 or anti-IGF-2 antibody. After extraction, residual, non-targeted species were removed from the MSIA-Tips by rinsing with: 5×200 μL HBS; 3×200 μL H₂O; 3×200 μL 20:80 ACN:H₂O; and 3×200 μL H₂O. Retained species were eluted from the MSIA-Tips and prepared for MALDI-TOF MS by drawing ˜4 μL of the MALDI matrix α-cyano-4-hydroxycinnamic acid (ACCA; dissolved in 1:2 ACN:H₂O, 0.03% TFA) into the tip and expelling/depositing the matrix/eluate mixture directly onto a MALDI-TOF MS target. MALDI-TOF MS then proceeded as generally practiced.

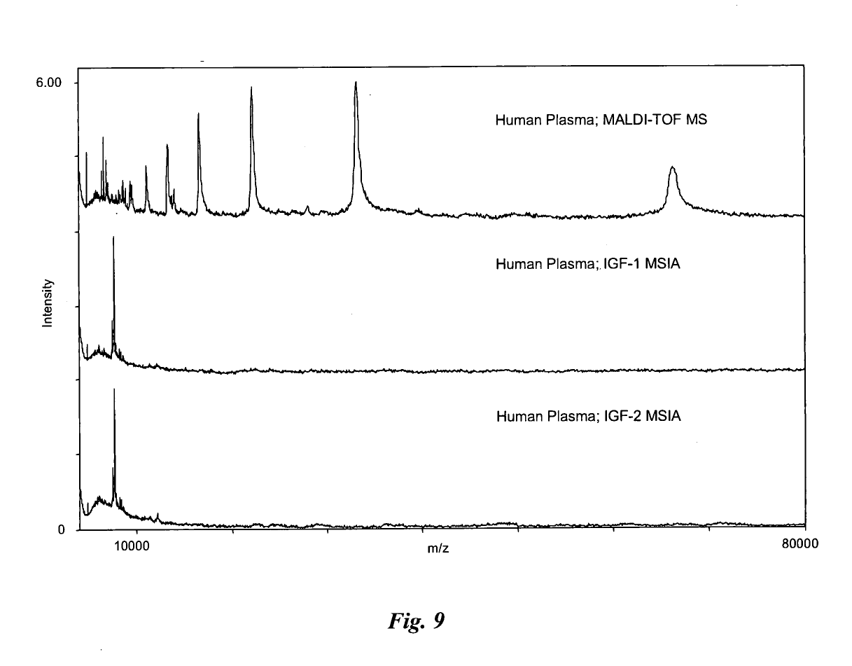

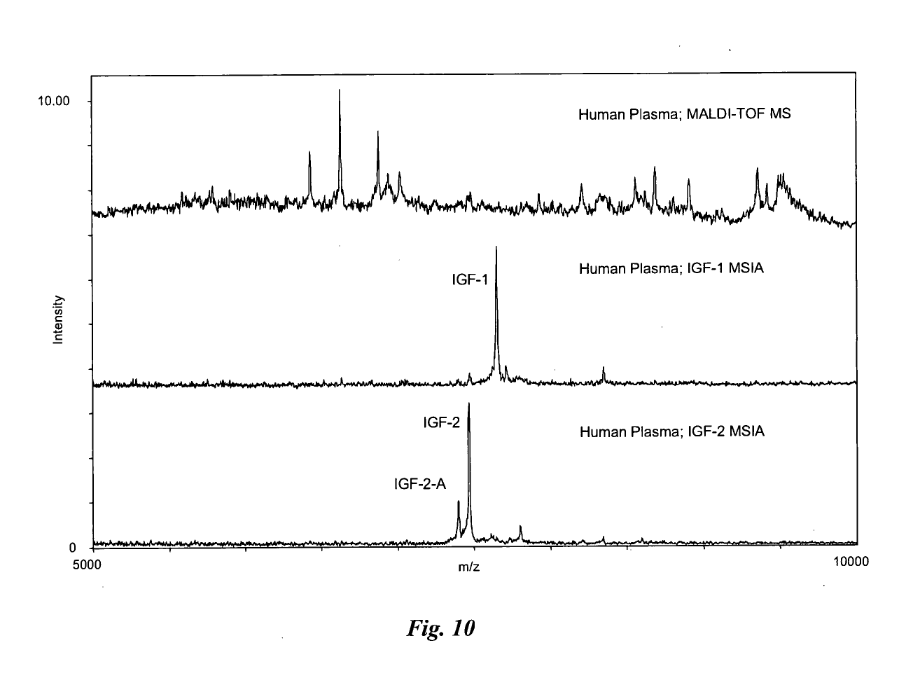

FIGS. 9 and 10 show results typical of the MSIA analysis of IGF (1 or 2) from plasma. Shown are two different mass ranges (4-80 kDa; and 6-9 kDa) of three spectra taken from the same plasma sample. The first spectrum was obtained through direct MALDI-TOF MS analysis of the plasma sample—i.e., without the benefit of MSIA preparation. The spectrum is dominated by signals derived from serum albumin and other high-abundance proteins, with no signals observed for either IGF-1 or IGF-2 (see FIG. 10). Subsequent MSIA analyses of the plasma sample yielded spectra dominated by either IGF-1 (MW=7,648.7) or IGF-2 (MW=7,469.4), dependent on which IGF was targeted, and that were largely free of artifacts/interferences. These data, taken from a single individual, indicate genetic homozygousity for both wild-type IGF-1 and IGF 2 (by observation of signals at m/z values expected for the wild-type proteins, and the lack of peak splitting which would be indicative of a single nucleotide polymorphism (SNP) present in one copy of either gene). However, the signal for IGF-2 is accompanied by a second signal at m/z=7,393 (˜77 Da less than the IGF-2 signal), which is most easily explained by the presence of a posttranslationally truncated version of the IGF 2 that lacks the N-terminal Alanine residue.

### EXAMPLE 9

### Qualitative Analysis of IGF-1 and IGF-2 using MSIA (Single Assay)

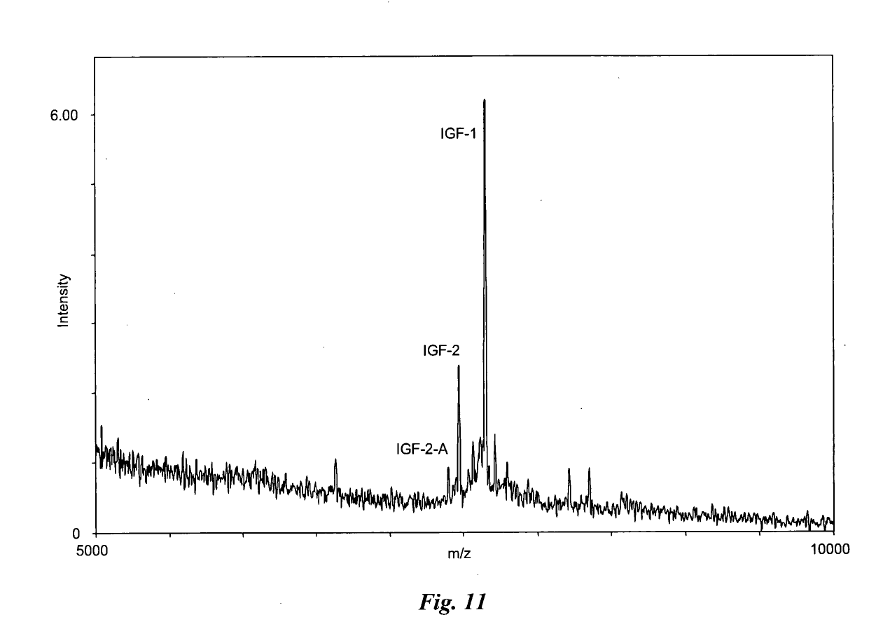

MSIA-Tips were prepared as described in EXAMPLE 7, with the exception of using a mixture of anti-IGF-1 and anti-IGF-2 IgG in place of the singe antibody solutions. Subsequently, plasma, prepared as described in EXAMPLE 8, was analyzed for both IGF-1 and IGF-2 in a single analysis by using the MSIA-Tips that target both of the growth hormones. FIG. 11 shows the results of the IGF 1 and IGF 2 multiplex assay. Similar to the results described in EXAMPLE 8, IGF 1, IGF-2 and IGF-2-A are observed (as homozygous species) at mass values within 0.05% of those theoretically calculated.

### EXAMPLE 10

### Quantitative Analysis of IGF 1 (Calibration Curve)

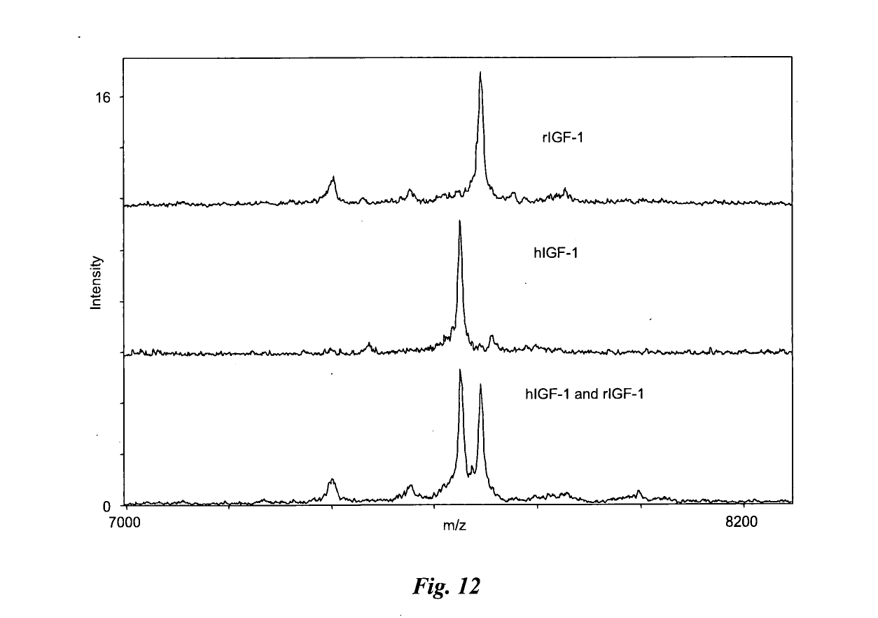

The IGF-1 MSIA analyses were made rigorously quantitative by inclusion of an internal reference standard (IRS) into the analysis, and the generation of a calibration curve (working curve) that equates concentration (of endogenous IGF-1) with relative signal intensity (human IGF-1/IRS). Because of similarity in amino acid sequence, cross-reactivity with anti-human IGF-11 antibody, and a resolvable mass difference, rat IGF-1 (rIGF-1) present in rat plasma was used as an IRS. FIG. 12 shows an anti-IGF-1 MSIA spectrum taken from rat and human plasma, and a human/rat plasma mixture. Observed in the spectra are dominant signal from the corresponding IGF-1s, with sufficient resolution between the two species for accurate quantification of the human IGF-1(hIGF-1).

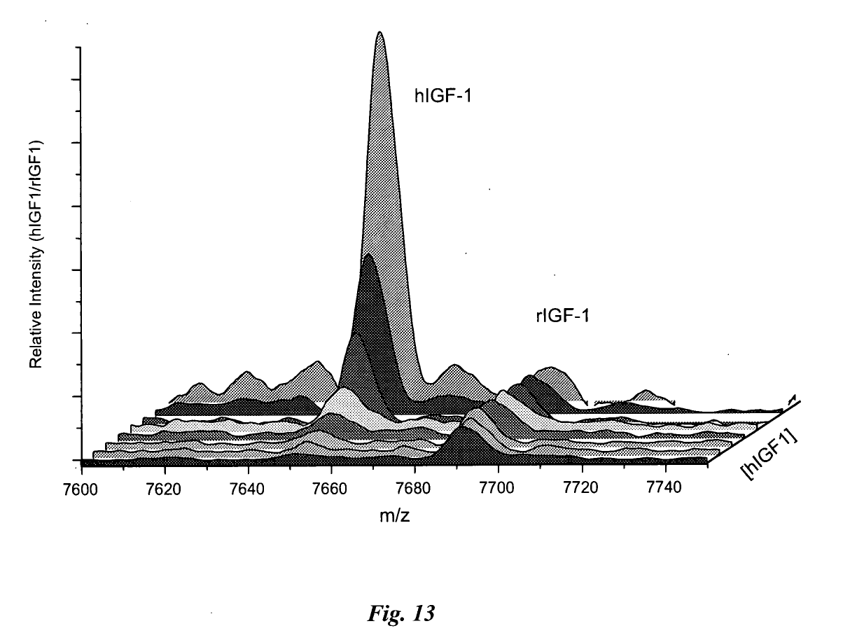

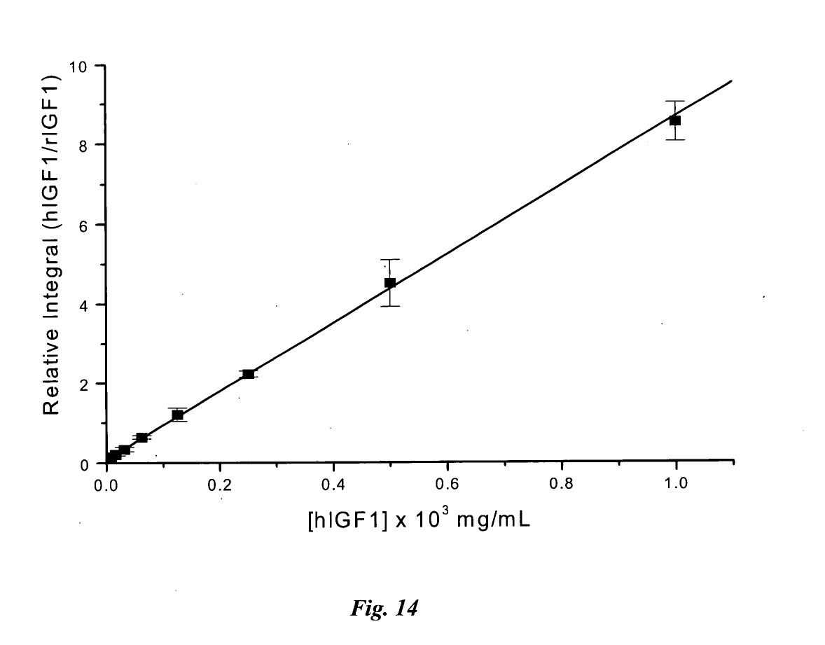

Samples for generating a quantitative calibration curve for hIGF-1 were prepared as described in EXAMPLE 8, except now each sample included a 20 μL aliquot of rat plasma (note: the initial 60 μL aliquot of HBS was reduced to 40 μL in this procedure) and the 40 μL human plasma sample was substituted with a 40 μL aliquot of purified hIGF-1 standard). Eight hIGF-1 standards at (equivalent plasma) concentrations ranging from 0.008 to 1 μg/mL were prepared for analysis. Both hIGF-1 and rIGF-1 were co-extracted from the samples using anti-IGF-1 MSIA-Tips prepared as described in EXAMPLE 7 and prepared for mass analysis as described in EXAMPLE 8. FIG. 13 shows the mass spectra taken from each of the standard sample. The spectra are normalized (y-axis) to the signal of the rIGF-1 and show a progressive increase in hIGF-1 signal with concentration. FIG. 14 shows the response (working) curve relating hIGF-1 concentration to normalized signal response. Each data point is the average of five 200-laser shot mass spectra taken from each standard sample. The y-axis error bars indicate the standard error of each data point. A linear relationship (R^{2=0.9998}) is observed over the concentration range under investigation.

### EXAMPLE 11

### Quantitative Analysis of IGF-1 (Population Screening)

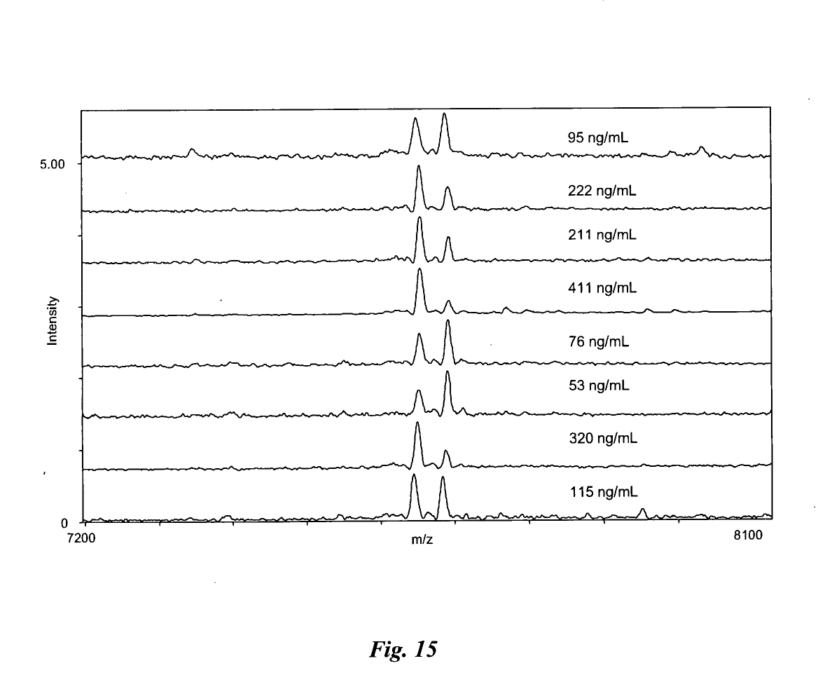

The quantitative IGF-1 MSIA was applied to eight individuals (3 females and 5 males; age range 28-46 years old) to determine to concentration of IGF-1 present in plasma. FIG. 15 shows the resulting mass spectra. Qualitative variants were not observed in any of the individuals. Importantly, the rIGF-1 reference species was adequately resolved from the hIGF-1 signal. IGP-1 concentrations were determined to range between 53-411 μg/mL in the eight individual participating in the study.

The present invention and the results shown in the Figures and Examples clearly demonstrate the usefulness of BIA/MS and MSIA in the analysis of insulin-like growth factors and their variants present in various biological fluids as well as the need for methods, devices and kits to expedite and enable the use of BIA/MS and MSIA in the analysis of large numbers of individuals.

1. A method for qualitatively and quantitatively detecting target biomolecules and their variants that are present in a biological fluid comprising the steps of:
providing a microfluidic chip having at least one site derivatized with at least one functional group susceptible to covalent ligand attachment;
immobilizing a ligand to said at least one site;
delivering a biological fluid sample containing at least one analyte over the site;
quantifying the binding of the analyte to the immobilized ligand at the site via surface plasmon resonance (SPR);
converting the site into a matrix-assisted laser desorption/ionization (MALDI) target via application of a MALDI matrix; and
subjecting the site to a matrix-assisted laser desorption/ionization time-of-flight (MALDI TOF) mass spectrometry.

2. The method of claim 1 wherein the biomolecule is an insulin-like growth factor.

3. The method of claim 2 wherein IGF-1 and IGF-2 are simultaneously detected.
